(19) United States
(12) Patent Application Publication
(10) Pub. No.: US 2017/0102389 A1
(43) Pub. Date: Apr. 13, 2017

(54) NEW BIOMARKER FOR OUTCOME IN AML PATIENTS

(71) Applicants: INSERM (INSTITUT NATIONAL DE LA SANTÉ ET DE LA RECHERCHE MÉDICALE), Paris (FR); INSTITUT JEAN PAOLI & IRENE CALMETTES, Marseille (FR); CENTRE NATIONAL DE LA RECHERCHE SCIENTIFIQUE (CNRS), Paris (FR); UNIVERSITÉ D'AIX MARSEILLE, Marseille 07 (FR)

(72) Inventors: Daniel OLIVE, Marseille (FR); Cyril FAURIAT, Marseille (FR); Anne-Sophie CHRETIEN, Marseille (FR); Christine ARNOULET, Marseille (FR); Didier BLAISE, Marseille Cedex (FR)

(21) Appl. No.: 15/128,654

(22) PCT Filed: Mar. 26, 2015
(86) PCT No.: PCT/EP2015/056619

§ 371 Date: Sep. 23, 2016

(30) Foreign Application Priority Data
Mar. 26, 2014 (EP) 14305435.1

(51) Int. Cl. G01N 33/574; C12Q 1/68

(52) U.S. Cl. CPC G01N 33/57426; C12Q 1/6886; G01N 33/57492; C12Q 2600/118; C12Q 2600/158; G01N 2800/52; G01N 2333/70596

(57) ABSTRACT

The present invention relates to a method for predicting the survival time of a patient suffering from acute myeloid leukemia (AML) comprising i) determining in a sample obtained from the patient the expression level of NKp46 ii) comparing the expression level determined at step i) with its predetermined reference value and iii) providing a good prognosis when the expression level determined at step i) is higher than its predetermined reference value, or providing a bad prognosis when the expression level determined at step i) is lower than its predetermined reference value.

### FIELD OF THE INVENTION

The present invention relates to a method for predicting the survival time of a patient suffering from acute myeloid leukemia (AML) comprising i) determining in a sample obtained from the patient the expression level of NKp46 ii) comparing the expression level determined at step i) with its predetermined reference value and iii) providing a good prognosis when the expression level determined at step i) is higher than its predetermined reference value, or providing a bad prognosis when the expression level determined at step i) is lower than its predetermined reference value.

### BACKGROUND OF THE INVENTION

Acute myeloid leukemia (AML), also known as acute myelogenous leukemia or acute non lymphocytic leukemia (ANLL), is a cancer of the myeloid line of blood cells, characterized by the rapid growth of abnormal white blood cells that accumulate in the bone marrow and interfere with the production of normal blood cells. AML is the most common acute leukemia affecting adults, and its incidence increases with age. Although AML is a relatively rare disease, accounting for approximately 1.2% of cancer deaths in the United States, its incidence is expected to increase as the population ages.

The symptoms of AML are caused by replacement of normal bone marrow with leukemic cells, which causes a drop in red blood cells, platelets, and normal white blood cells. These symptoms include fatigue, shortness of breath, easy bruising and bleeding, and increased risk of infection. Several risk factors and chromosomal abnormalities have been identified, but the specific cause is not clear. As an acute leukemia, AML progresses rapidly and is typically fatal within weeks or months if left untreated.

AML has several subtypes; treatment and prognosis varies among subtypes. Five-year survival varies from 15-70%, and relapse rate varies from 33-78%, depending on subtype. AML is treated initially with chemotherapy aimed at inducing a remission; patients may go on to receive additional chemotherapy or a hematopoietic stem cell transplant. Recent research into the genetics of AML has resulted in the availability of tests that can predict which drug or drugs may work best for a particular patient, as well as how long that patient is likely to survive (Dohner et al., 2005 and Dohner et al., 2010). For example, usual parameters used in clinical practice to evaluate the prognosis of AML patients prior to HSCT are the Disease Relapse Index (DRI) (Armand et al. Blood 2012) and the Hematopoietic Cell Transplantation Comorbidity Index (HCT-CI) (Sorror et al. 2005 and Sorror et al. JCO 2007).

However, there is still a need for a good and robust biomarker to determine the outcome of a patient suffering from AML and who received a hematopoietic stem cell transplant.

### SUMMARY OF THE INVENTION

Knowing that NK cells receptors (NKp30, NKp44 and NKp46) specifically expressed by NK cells, are major determinants of NK cell functionality and are involved in tumor immune surveillance, especially in acute myeloid leukemia (AML), the inventors assess the significance of NKp46 expression at diagnosis in patients with hematopoietic stem cell transplantation (HSCT).

NKp46 expression was prospectively assessed at diagnosis in 125 patients with AML (with or without allograft) and post graft outcome was evaluated with regard to NKp46 expression. According to the invention, NKp46 expression predicts outcome of patients after HSCT. In other terms, classification of patients into 2 groups according to NKp46 expression at diagnosis (NKp46^{dull} and NKp46^{bright}) defines a group with high risk of relapse and poor clinical outcome (NKp46^{dull} phenotype) and a group with low risk of relapse and favourable clinical outcome (NKp46^{bright} phenotype).

Compared to the DRI and the HCT-CI, the use of NKp46 expression as biomarker for the outcome of AML presents the following advantages:
NKp46 expression is evaluable early at diagnosis. In others words, when the patient arrive to the hospital to do the AML diagnosis, NKp46 expression can be evaluate at the same time;
Patients are classified into 2 categories (NKp46bright or NKp46dull), which facilitates clinical decision making; and
NKp46 evaluation is cost-effective.

Thus, the present invention relates to a method for predicting the survival time of a patient suffering from acute myeloid leukemia (AML) comprising i) determining in a sample obtained from the patient the expression level of NKp46 ii) comparing the expression level determined at step i) with its predetermined reference value and iii) providing a good prognosis when the expression level determined at step i) is higher than its predetermined reference value, or providing a bad prognosis when the expression level determined at step i) is lower than its predetermined reference value.

### DETAILED DESCRIPTION OF THE INVENTION

A first aspect of the invention relates to a method for predicting the survival time of a patient suffering from acute myeloid leukemia (AML) comprising i) determining in a sample obtained from the patient the expression level of NKp46 ii) comparing the expression level determined at step i) with its predetermined reference value and iii) providing a good prognosis when the expression level determined at step i) is higher than its predetermined reference value, or providing a bad prognosis when the expression level determined at step i) is lower than its predetermined reference value.

In another embodiment, the invention relates to a method for predicting the overall survival (OS) of a patient suffering from acute myeloid leukemia (AML) comprising i) determining in a sample obtained from the patient the expression level of NKp46 ii) comparing the expression level determined at step i) with its predetermined reference value and iii) providing a good prognosis when the expression level determined at step i) is higher than its predetermined reference value, or providing a bad prognosis when the expression level determined at step i) is lower than its predetermined reference value

In another embodiment, the invention relates to a method for predicting the progression free survival (PFS) of a patient suffering from acute myeloid leukemia (AML) comprising i) determining in a sample obtained from the patient the expression level of NKp46 ii) comparing the expression level determined at step i) with its predetermined reference value and iii) providing a good prognosis when the expression level determined at step i) is higher than its predetermined reference value, or providing a bad prognosis when the expression level determined at step i) is lower than its predetermined reference value

As used herein, the term “Overall survival (OS)” denotes the percentage of people in a study or treatment group who are still alive for a certain period of time after they were diagnosed with or started treatment for a disease, such as AML (according to the invention). The overall survival rate is often stated as a five-year survival rate, which is the percentage of people in a study or treatment group who are alive five years after their diagnosis or the start of treatment.

As used herein, the term “Progression Free Survival (PFS)” denotes the length of time after primary treatment for a cancer ends that the patient survives without any signs or symptoms of that cancer or without disease progression.

As used herein, the term “Good Prognosis” denotes a patient with significantly enhanced probability of survival after treatment.

In a particular embodiment, patient suffering from acute myeloid leukemia (AML) has been treated by allograft.

As used herein the term “allograft” denotes a patient who has been treated by hematopoietic stem cell transplantation (HSCT). According to the term allograft, hematopoietic stem cells come from a donor related or not to the recipient but of the same species.

Thus, the invention also relates to a method for predicting the survival time of a patient suffering from acute myeloid leukemia (AML) and treated by allograft comprising i) determining in a sample obtained from the patient the expression level of NKp46 ii) comparing the expression level determined at step i) with its predetermined reference value and iii) providing a good prognosis when the expression level determined at step i) is higher than its predetermined reference value, or providing a bad prognosis when the expression level determined at step i) is lower than its predetermined reference value.

As used herein and according to all aspects of the invention, the term “NKp46” denotes a receptor of the natural cytotoxicity receptors (NCRs) family. NKp46 is a triggering receptor expressed on the plasmatic membrane of NK cells, also known as CD335, or NCR1.

As used herein and according to all aspects of the invention, the term “sample” denotes, blood, peripheral-blood, serum, plasma or purified NK cells.

Measuring the expression level of NKp46 can be done by measuring the gene expression level of NKp46 or by measuring the level of the protein NKp46 and can be performed by a variety of techniques well known in the art.

Typically, the expression level of a gene may be determined by determining the quantity of mRNA. Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) amino naphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Puhlication No. 2003/0165951 as well as PCT Puhlication No. 99/26299 (puhlished May 27, 1999). Separate populations of semiconductor nanocrystals can he produced that are identifiable based on their different spectral characteristics. For example, semiconductor nanocrystals can he produced that emit light of different colors hased on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlshad, Calif.).

Additional labels include, for example, radioisotopes (such as 3H), metal chelates such as DOTA and DPTA chelates of radioactive or paramagnetic metal ions like Gd3+, and liposomes.

Detectable labels that can he used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can he used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Puhlication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 0.1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will be appreciated by those of skill in the art that by appropriately selecting labelled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

Expression level of a gene may be expressed as absolute expression level or normalized expression level. Typically, expression levels are normalized by correcting the absolute expression level of a gene by comparing its expression to the expression of a gene that is not a relevant for determining the cancer stage of the patient, e.g., a housekeeping gene that is constitutively expressed. Suitable genes for normalization include housekeeping genes such as the actin gene ACTB, ribosomal 18S gene, GUSB, PGK1, TFRC, GAPDH, GUSB, TBP and ABL1. This normalization allows the comparison of the expression level in one sample, e.g., a patient sample, to another sample, or between samples from different sources.

Predetermined reference values used for comparison may comprise “cut-off” or “threshold” values that may be determined as described herein. Each reference (“cut-off”) value for NKp46 expression may be predetermined by carrying out a method comprising the steps of

a) providing a collection of samples from patients suffering of AML treated or not by allograft;

b) determining the expression level of NKp46 for each sample contained in the collection provided at step a);

c) ranking the tumor tissue samples according to said expression level

d) classifying said samples in pairs of subsets of increasing, respectively decreasing, number of members ranked according to their expression level,

e) providing, for each sample provided at step a), information relating to the actual clinical outcome for the corresponding cancer patient (i.e. the duration of the progression free survival (PFS) or the overall survival (OS) or both);

f) for each pair of subsets of samples, obtaining a Kaplan Meier percentage of survival curve;

g) for each pair of subsets of samples calculating the statistical significance (p value) between both subsets

h) selecting as reference value for the expression level, the value of expression level for which the p value is the smallest.

For example the expression level of NKp46 has been assessed for 100 AML samples of 100 patients. The 100 samples are ranked according to their expression level. Sample 1 has the best expression level and sample 100 has the worst expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding AML patient, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels.

In routine work, the reference value (cut-off value) may be used in the present method to discriminate AML samples and therefore the corresponding patients.

Kaplan-Meier curves of percentage of survival as a function of time are commonly used to measure the fraction of patients living for a certain amount of time after treatment and are well known by the man skilled in the art.

The man skilled in the art also understands that the same technique of assessment of the expression level of a gene should of course be used for obtaining the reference value and thereafter for assessment of the expression level of a gene of a patient subjected to the method of the invention.

Such predetermined reference values of expression level may be determined for any gene defined above.

According to the invention, the level of the protein NKp46 may also be measured and can be performed by a variety of techniques well known in the art.

Typically protein concentration may be measured for example by capillary electrophoresis-mass spectroscopy technique (CE-MS) or ELISA performed on the sample.

Detection of protein concentration in the sample may also be performed by measuring the level of the protein NKp46. In the present application, the “level of protein” or the “protein level expression” means the quantity or concentration of said protein. In another embodiment, the “level of protein” means the level of NKp46 protein fragments. In still another embodiment, the “level of protein” means the quantitative measurement of the protein NKp46 expression relative to a negative control.

Such methods comprise contacting a sample with a binding partner capable of selectively interacting with proteins present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, capillary electrophoresis-mass spectroscopy technique (CE-MS). etc. The reactions generally include revealing labels such as fluorescent, chemioluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against the proteins to be tested. A sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule is added. The secondary binding molecule is allowed to react with any captured sample marker protein, the plate is washed and the presence of the secondary binding molecule is detected using methods well known in the art.

Methods of the invention may comprise a step consisting of comparing the proteins and fragments concentration in circulating cells with a control value. As used herein, “concentration of protein” refers to an amount or a concentration of a transcription product, for instance the protein NKp46. Typically, a level of a protein can be expressed as nanograms per microgram of tissue or nanograms per milliliter of a culture medium, for example. Alternatively, relative units can be employed to describe a concentration. In a particular embodiment, “concentration of proteins” may refer to fragments of the protein NKp46. Thus, in a particular embodiment, fragment of NKp46 protein may also be measured.

In a particular embodiment, the detection of the level of NKp46 can be performed by flow cytometry. When this method is used, the method consists of determining the amount of NKp46 expressed on NK cells. According to the invention and the flow cytometry method, when the florescence intensity is high or bright, the level of NKp46 express on NK cells is high and thus the expression level of NKp46 is high and when the florescence intensity is low or dull, the level of NKp46 express on NK cells is low and thus the expression level of NKp46 is low.

Thus, according to the invention when the expression level of NKp46 is high the prognosis of the patient suffering from AML and treated by graft is good and when the expression level of NKp46 is low the prognosis of the patient suffering from AML and treated by graft is bad.

In another embodiment, the extracellular part of the NKp46 protein is detected.

In a further embodiment of the invention, methods of the invention comprise measuring the expression level of at least one further biomarker or prognostic score.

The term “biomarker”, as used herein, refers generally to a cytogenetic marker, a molecule, the expression of which in a sample from a patient can be detected by standard methods in the art (as well as those disclosed herein), and is predictive or denotes a condition of the subject from which it was obtained.

Various validated prognostic biomarkers or prognostic scores may be combined to NKp46 in order to improve methods of the invention and especially some parameters such as the specificity (see for example Cornelissen et al. 2012).

For example, the other biomarkers may be selected from the group of AML biomarkers consisting of cytogenetics markers (like t(8;21), t(15;17), inv(16) see for example Grimwade et al., 2010 or Byrd et al., 2002), lactate dehydrogenase (see for example Haferlach et al 2003), FLT3, NPM1, CEBPa (see for example Schnittger et al., 2002, Dohner et al., 2010). The prognostic scores that may be combined to NKp46 may be for example the Hematopoietic Cell Transplantation Comorbidity Index (HCT-CI) (Sorror et al 2005), the comorbidity and disease status (Sorror et al 2007) or the disease risk index (DRI) (Armand et al 2012).

In a particular embodiment, the invention relates to a method for predicting the survival time of a patient suffering from acute myeloid leukemia (AML) comprising i) determining in a sample obtained from the patient the expression level of NKp46 and the HCT-CI ii) comparing the expression level and the HCT-CI score determined at step i) with its predetermined reference value and reference score and iii) providing a good prognosis when the expression level determined at step i) is higher than its predetermined reference value or when the HCT-CI is equal to 0, or providing a bad prognosis when the expression level determined at step i) is lower than its predetermined reference value and the HCT-CI is superior or equal to 1.

A further object of the invention relates to kits for performing the methods of the invention, wherein said kits comprise means for measuring the expression level of NKp46 in the sample obtained from the patient.

The kits may include probes, primers macroarrays or microarrays as above described. For example, the kit may comprise a set of probes as above defined, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards. Alternatively the kit of the invention may comprise amplification primers that may be pre-labelled or may contain an affinity purification or attachment moiety. The kit may further comprise amplification reagents and also other suitably packaged reagents and materials needed for the particular amplification protocol.

The present invention also relates to NKp46 as a biomarker for outcome of AML patients.

The present invention also relates to NKp46 as a biomarker for post-graft outcome of AML patients.

The invention will be further illustrated by the following figures and examples. However, these examples and figures should not be interpreted in any way as limiting the scope of the present invention.

### FIGURES

FIG. 1: Threshold determination for NKp46 expression on NK cells.

Panels A and B show distribution histograms of NKp46 mean fluorescence intensity (MFI) ratio (NKp46 MFI/isotype control MFI) in patients with AML at diagnosis (A) and healthy volunteers (B). Panels C and D show the relation between risk groups and NKp46 expression at diagnosis.

FIG. 2: Kaplan Meier curves of overall survival (A, C) and progression-free survival (B, D) by NKp46 expression at diagnosis. (A, B) non-allografted patients; (C, D) patients with allogeneic HSCT. HR, hazard ratio.

FIG. 3: Kaplan Meier curves of overall survival (A, C) and progression-free survival (B, D) by Disease Relapse Index (DRI) (A, B) and Hematopoietic Cell Transplantation Comorbidity Index (HCT-CI) (C, D).

FIG. 4: Kaplan Meier curves of overall survival (A) and progression-free survival (B) after combination of the HCT-CI and NKp46 expression. Patients were classified in two groups. The first group is defined as patients with HCT-CI=0 or high NKp46 expression at diagnosis. The second group is defined as patients with HCT-CI≧1 and low NKp46 expression at diagnosis.

TABLE 1
Comparison of HCT-CI with NKp46 expression

| | | | HCT-CI + |
|---|---|---|---|
| Classification | HCT-CI | NKp46 | NKp46 |
| Sensitivity | 100% | 95% | 94% |
| Specificity | 29% | 38% | 54% |
| Negative predictive value | 100% | 90% | 93% |
| Positive predictive value | 50% | 55% | 59% |
| % well-classified patients | 59% | 63% | 70% |
| % patients in the good prognosis group | 19% | 22% | 35% |

TABLE 2
Multivariate analysis

| | Multivariate HR for OS | | | Multivariate HR for PFS | | |
|---|---|---|---|---|---|---|
| Variable | HR | 95% CI | P | HR | 95% CI | P |
| Status at | | | | | | |
| graft | | | | | | |
| CR1 | Reference | | | Reference | | |
| CR2, PR, | 3.37 | 1.45 to | 0.004 | 2.86 | 1.26 to | 0.013 |
| Refractory | | 7.77 | | | 6.44 | |
| HCT-CI | | | | | | |
| 0 | Reference | | | Reference | | |
| ≧1 | 5.21 | 0.69 to | 0.109 | 2.62 | 0.60 to | 0.196 |
| | | 39.17 | | | 11.25 | |
| NKp46 | | | | | | |
| expression | | | | | | |
| High | Reference | | | Reference | | |
| Low | 6.13 | 0.82 to | 0.077 | 7.46 | 1.00 to | 0.049 |
| | | 45.68 | | | 55.34 | |

CR: complete remission; HCT-CI: Hematopoietic cell transplantation comorbidity index; PR: partial remission; HR: Hazard ratio

### Example

### Material & Methods

Patients

Peripheral-blood samples were obtained from AML patients at diagnosis before induction chemotherapy and from healthy volunteers. All participants gave written informed consent in accordance with the Declaration of Helsinki Patients above 65 years old at diagnosis were excluded. The entire research procedure was approved by the ethical review board (Institut Paoli-Calmettes Marseille, France).

Flow Cytometry

A FACS Canto II (BD Biosciences, San Jose, Calif.) and FACS Diva Software (BD Biosciences) were used for flow cytometry. Isotypic controls were mouse immunoglobulin G conjugated to fluorescein isothiocyanate (FITC), phycoerythrin (BD Biosciences) or phycoerythrin-cyanine 5 (kind gift of Beckman-Coulter, Marseille, France). NK cells from whole blood EDTA were immunostained with fluorescein isothiocyanate (FITC)-conjugated anti-CD3, Phycoerythrin cyanin 7 (PC7)-conjugated anti-CD56 and allophycocyanin (APC)-conjugated anti-CD45 antibodies. Triggering receptor expression NKp30 and NKp46 were measured with phycoerythrin (PE)- and Phycoerythrin-Cyanine 5.1 (PC5)-conjugated monoclonal antibodies, respectively (kind gift of Beckman-Coulter, Marseille, France). Red blood cells were lysed with BD FACS Lysing solution (BD Biosciences) before data acquisition.

Statistical Analyses

Statistical analyses were carried out using Graph Pad Prism (Graph Pad Software, San Diego, Calif.). In all statistical analyses, the limit of significance was set at P<0.05. The threshold for NKp46 expression was determinated by dispersion criteria and normality of distributions, assessed by the d'Agostino and Pearson normality test and the Kernel density estimation.

The cohort was divided into two groups according to NKp46 expression at diagnosis. For allografted patients, primary endpoint was overall survival (OS), defined as the time between HSCT and death irrespective of cause, or censored at last follow-up. Secondary endpoint was progression-free survival (PFS) defined as the time from date of HSCT to relapse, progression or death irrespective of cause or censored at last follow-up. For non-allografted patients, OS was defined as the time from date of diagnosis to death or censored at last follow-up and PFS was defined as the time from date of complete remission (CR) to relapse or death or censored at last follow-up. Survival distribution was estimated by the Kaplan-Meier method and the significance of differences between survival rates was ascertained by the log-rank test (univariate analysis).

Results

Patients

From December 2007 to December 2011, 118 patients were prospectively assessed for baseline NKp46 expression at diagnosis. Sixtyfour (54%) of these patients received allogeneic hematopoietic stem cell transplantation (HSCT). The median follow-up was 35.1 months (range: [34.1-41.6]).

Threshold Determination

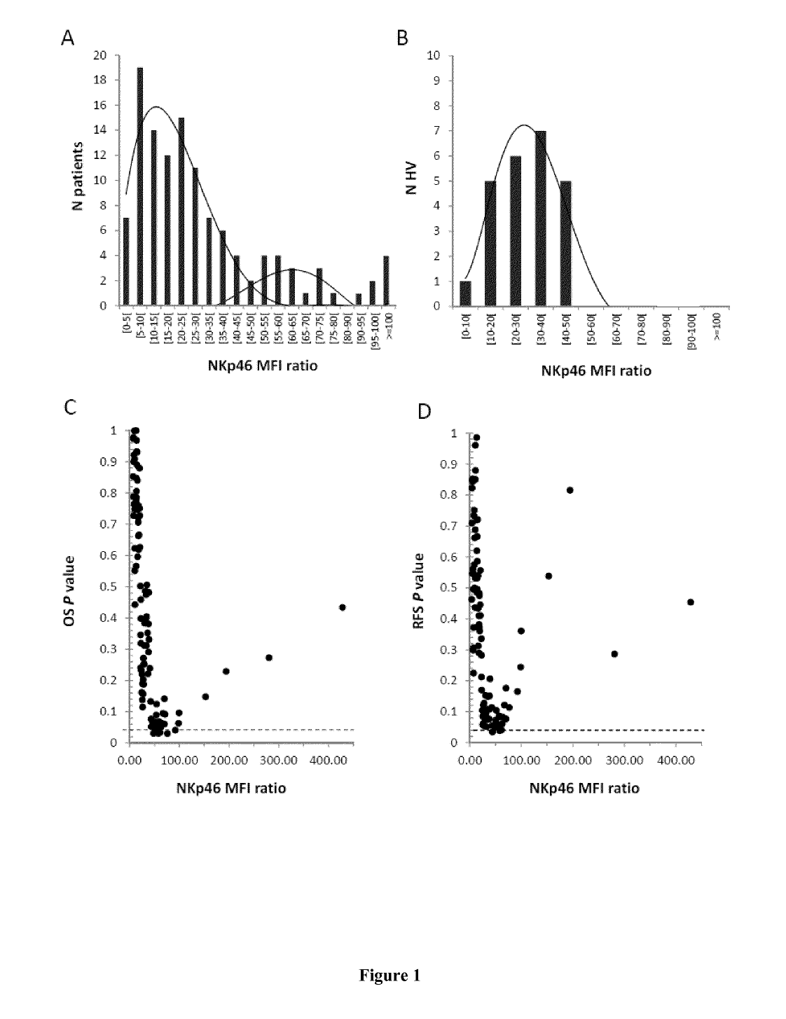

Patients were classified into two groups according to NKp46 mean fluorescence intensity (MFI) ratio (NKp46 MFI/isotype control MFI). The dichotomy between NKp46^{dull} and NKp46^{bright} patients was based on dispersion criteria of the population. Inter individual variability of NKp46 expression in AML patients (FIG. 1A) and healthy volunteers (HV) (FIG. 1B) was represented on a distribution histogram. The distribution was found to be unimodal for HV and bimodal for AML patients. In FIG. 1A, the threshold between these 2 peaks was found to be NKp46 MFI ratio=43.69. The distribution of NKp46 expression was found to be a juxtaposition of two Gaussian distributions. The normality of these 2 peaks was evidenced by the d'Agostino and Pearson normality test and confirmed by the Kernel density estimation. This threshold was found to be above the 90th percentile of HV (FIG. 1B). In addition, this threshold was the most discriminant threshold for overall survival (OS) and progression-free survival (PFS) (FIGS. 1C and 1D, respectively). We accordingly classified the patients in 2 distinct subgroups for survival analyses. In the total population, 22% (26/118) of patients were then classified in the NKp46^{bright} subgroup, and 78% (92/118) were classified in the NKp46^{dull} subgroup. Among allografted patients, 22% (12/54) of patients were classified in the NKp46^{bright} subgroup, and 78% (42/54) were classified in the NKp46^{dull} subgroup. This threshold is fixed for all the following calculations.

NKp46 at Diagnosis Predicts Post Graft Clinical Outcome.

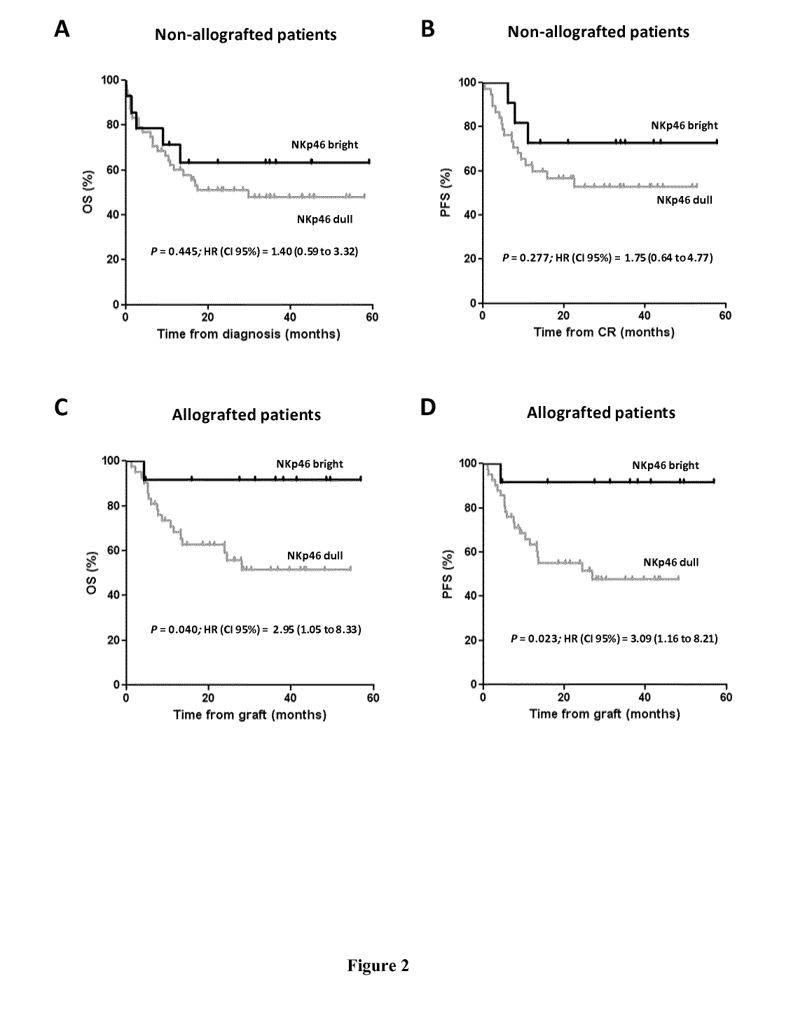

Kaplan-Meier curves of OS and PFS are shown in FIG. 2. For the non-allografted population, no significant difference was observed between patients with NKp46^{dull} and NKp46^{bright} phenotype in terms of OS and PFS (FIGS. 2A and 2B). In the group of allografted patients, patients with NKp46^{bright} phenotype at diagnosis had better OS (P=0.040; hazard ratio=2.95; 95% CI=[1.05-8.33]) (FIG. 2C) and PFS (P=0.023; hazard ratio=3.09; 95% CI=[1.16-8.21]) (FIG. 2D) than patients with NKp46^{dull} phenotype. Moreover, allografted patients with NKp46^{bright} phenotype had lower death probabilities at 2 years compared with patients with NKp46^{dull} phenotype (10% vs 47%, respectively) as well as lower relapse probabilities at 2 years (0% vs 27%, respectively). To summarize, the clinical benefit of high NKp46 expression on NK cells at diagnosis was specifically observed in the subgroup of allogeneic HSCT patients. No clinical benefit was observed for non-allografted patients with high NKp46 expression compared to non-allografted patients with low NKp46 expression. Taken together, these data suggest that NKp46 expression is a predictive biomarker of post graft outcome.

Sensitivity, Specificity, Negative and Positive Predictive Values of NKp46 Expression

For these calculations, we considered the actuarial 2-year survival and relapse rates for patients with allogeneic HSCT according to NKp46 expression. The sensitivity and the negative predictive values were found to be 95 and 90%, respectively. However, the specificity and the positive predictive value were found to be poor (38 and 55%, respectively).

Comparison with Performances of the Prognostic Scores Used in Clinical Practice

Two scores are commonly used in clinical practice: the Disease Relapse Index (DRI) and the Hematopoietic Cell Transplantation Comorbidity Index (HCT-CI). We evaluated the performance of these scores on our cohort in order to compare their performance with the classification according to NKp46 expression.

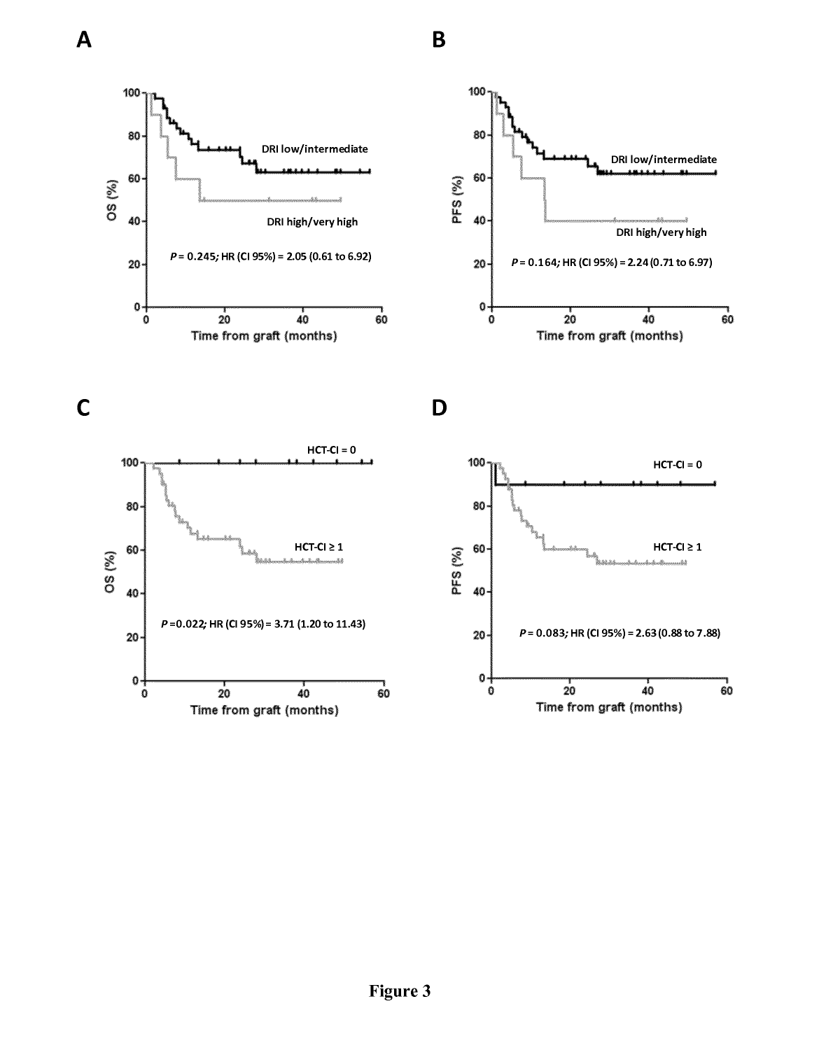

In the case of DRI, the difference between patients in the DRI low/DRI intermediate was found to be non significant in terms of OS and PFS (FIGS. 3A and 3B, respectively). However, the trends in terms of median survival were found to be consistent with previously published results on larger cohorts of patients (Armand et al. Blood 2012), thus suggesting that the lack of significance is due to the small effective of our cohort.

In the case of HCT-CI, the difference between patients with HCT-CI=0 and patients with HCT-CI≧1 was significant in terms of OS (P=0.022; HR (CI 95%)=3.71 (1.20 to 11.43)) and non significant in terms of PFS (P=0.083; HR (CI 95%)=2.63 (0.88 to 7.88)) (FIGS. 3C and 3D, respectively). Thus, the classification of patients according to NKp46 expression was found to be better in terms of OS and PFS compared to classification according to DRI and HCT-CI.

Combination of NKp46 Expression with HCT-CI

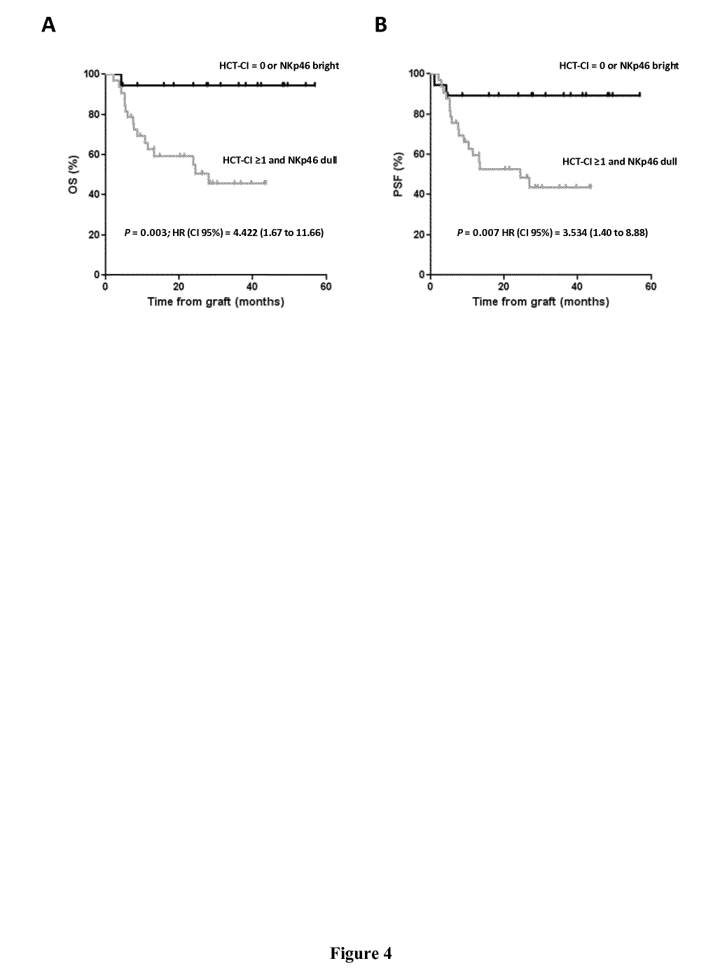

There was a poor overlap between NKp46 expression and HCT-CI: among the 10 patients with HCT-CI=0 and the 12 patients with NKp46bright phenotype, only 3 patients had both HCT-CI=0 and NKp46bright phenotype. Thus, we combined both classifications as follow. Patients were divided into 2 groups. The first group was defined as patients with HCT-CI=0 or high NKp46 expression at diagnosis. The second group was defined as patients with HCT-CI≧1 and low NKp46 expression at diagnosis. The combination of these 2 classifications improved the results in terms of OS (P=0.003; HR (CI 95%)=4.422 (1.67 to 11.66)) and PFS (P=0.007; HR (CI 95%)=3.534 (1.40 to 8.88)) (FIG. 4). Combining these parameters allowed increasing the specificity with a limited impact on the sensitivity (Table 1). Moreover, combining these parameters allowed increasing the number of patients in the group of good prognosis (HCT-CI: 18%; NKp46 expression: 22%; HCT-CI+NKp46 expression 35%) (Table 1).

Confirmation of the Results:

Discriminating ability of our approach is currently prospectively challenged using an independent cohort of allografted AML patients. Results obtained with 10 patients evidenced that hazards ratios for OS and PFS (HR=3.71 and 3.83, respectively) are fully consistent with the results obtained on the previous dataset (with the first 54 patients).

When the 54 first patients and the others 10 patients are combined (for a total of 64 patients) hazards ratios for OS and PFS are respectively 3.02 and 3.14. These data suggest that NKp46 is a strong biomarker.

Multivariate Analysis (Data Obtained on the 64 Patients of the Cohort)

Multivariate Cox regression models were used to assess the predictive value of NKp46 expression while adjusting for the prognostic factors in the population (age at transplantation, donor HLA match, conditioning regimen, status at graft and HCT-CI), with stepwise selection at a 0.15 level (Table 2). Data from the training cohort and the validation set were pooled for these analyses. The multivariate analysis demonstrated that low NKp46 expression was significantly associated with reduced PFS (P=0.013), independent of other factors.

### REFERENCES

Throughout this application, various references describe the state of the art to which this invention pertains. The disclosures of these references are hereby incorporated by reference into the present disclosure.
Byrd John C., Krzysztof Mro'zek, Richard K. Dodge, Andrew J. Carroll, Colin G. Edwards, Diane C. Arthur, Mark J. Pettenati, Shivanand R. Patil, Kathleen W. Rao, Michael S. Watson, Prasad R. K. Koduru, Joseph O. Moore, Richard M. Stone, Robert J. Mayer, Eric J. Feldman, Frederick R. Davey, Charles A. Schiffer, Richard A. Larson, and Clara D. Bloomfield. Pretreatment cytogenetic abnormalities are predictive of induction success, cumulative incidence of relapse, and overall survival in adult patients with de novo acute myeloid leukemia: results from Cancer and Leukemia Group B (CALGB 8461). Blood, 15 Dec. 2002—volume 100, number 13.
Cornelissen J Jl, Gratwohl A, Schlenk R F, Sierra J, Bornhäuser M, Juliusson G, Råcil Z, Rowe J M, Russell N, Mohty M, Löwenberg B, Socié G, Niederwieser D, Ossenkoppele G J. The European LeukemiaNet AML Working Party consensus statement on allogeneic HSCT for patients with AML in remission: an integrated-risk adapted approach. Nat Rev Clin Oncol. 2012 October; 9(10):579-90. doi: 10.1038/nrclinonc.2012.150. Epub 2012 Sep. 4.
Döhner K l, Schlenk R F, Habdank M, Scholl C, Rucker F G, Corbacioglu A, Bullinger L, Fröhling S, Dohner H. Mutant nucleophosmin (NPM1) predicts favorable prognosis in younger adults with acute myeloid leukemia and normal cytogenetics: interaction with other gene mutations. Blood. 2005 Dec. 1; 106(12):3740-6. Epub 2005 Jul. 28.
Döhner H l, Estey E H, Amadori S, Appelbaum F R, Buchner T, Burnett A K, Dombret H, Fenaux P, Grimwade D, Larson R A, Lo-Coco F, Naoe T, Niederwieser D, Ossenkoppele G J, Sanz M A, Sierra J, Tallman M S, Löwenberg B, Bloomfield C D; European LeukemiaNet. Diagnosis and management of acute myeloid leukemia in adults: recommendations from an international expert panel, on behalf of the European LeukemiaNet. Blood. 2010 Jan. 21; 115(3):453-74. doi: 10.1182/blood-2009-07-235358. Epub 2009 Oct. 30.
Grimwade D l, Hills R K, Moorman A V, Walker H, Chatters S, Goldstone A H, Wheatley K, Harrison C J, Burnett A K; National Cancer Research Institute Adult Leukaemia Working Group. Refinement of cytogenetic classification in acute myeloid leukemia: determination of prognostic significance of rare recurring chromosomal abnormalities among 5876 younger adult patients treated in the United Kingdom Medical Research Council trials. Blood. 2010 Jul. 22; 116(3):354-65. doi: 10.1182/blood-2009-11-254441. Epub 2010 Apr. 12.
Haferlach Torsten, Claudia Schoch, Helmut Lo\ddot{}ffler, Winfried Gassmann, Wolfgang Kern, Susanne Schnittger, Christa Fonatsch, Wolf-Dieter Ludwig, Christian Wuchter, Brigitte Schlegelberger, Peter Staib, Albrecht Reichle, Uschi Kubica, Hartmut Eimermacher, Leopold Balleisen, Andreas Gru\ddot{}neisen, Detlef Haase, Carlo Aul, Jochen Karow, Eva Lengfelder, Bernhard Wo\ddot{}rmann, Achim Heinecke, Maria Cristina Sauerland, Thomas Bu\ddot{}chner, and Wolfgang Hiddemann. Morphologic Dysplasia in De Novo Acute Myeloid Leukemia (AML) Is Related to Unfavorable Cytogenetics but Has No Independent Prognostic Relevance Under the Conditions of Intensive Induction Therapy: Results of a Multiparameter Analysis From the German AML Cooperative Group Studies. Journal of Clinical Oncology, Vol 21, No 2 (January 15), 2003: pp 256-265.
Schnittger Susanne, Claudia Schoch, Martin Dugas, Wolfgang Kern, Peter Staib, Christian Wuchter, Helmut Löffler, Cristina Maria Sauerland, Hubert Serve, Thomas Bu\ddot{}chner, Torsten Haferlach, and Wolfgang Hiddemann. Analysis of FLT3 length mutations in 1003 patients with acute myeloid leukemia: correlation to cytogenetics, FAB subtype, and prognosis in the AMLCG study and usefulness as a marker for the detection of minimal residual disease. Blood, 1 Jul. 2002—volume 100, number 1.
Sorror M Ll, Maris M B, Storb R, Baron F, Sandmaier B M, Maloney D G, Storer B. Hematopoietic cell transplantation (HCT)-specific comorbidity index: a new tool for risk assessment before allogeneic HCT. Blood. 2005 Oct. 15; 106(8):2912-9. Epub 2005 Jun. 30.
Sorror M Ll, Sandmaier B M, Storer B E, Maris M B, Baron F, Maloney D G, Scott B L, Deeg H J, Appelbaum F R, Storb R. Comorbidity and disease status based risk stratification of outcomes among patients with acute myeloid leukemia or myelodysplasia receiving allogeneic hematopoietic cell transplantation. J Clin Oncol. 2007 Sep. 20; 25(27):4246-54. Epub 2007 Aug. 27.

1. A method for predicting survival time of a patient suffering from acute myeloid leukemia (AML) comprising i) determining in a sample obtained from the patient an expression level of NKp46 ii) comparing the expression level determined at step i) with its predetermined reference value and iii) providing a good prognosis when the expression level determined at step i) is higher than its predetermined reference value, or providing a bad prognosis when the expression level determined at step i) is lower than its predetermined reference value.

2. The method according to claim 1 wherein the patient suffering from acute myeloid leukemia (AML) has been treated by allograft.

3. The method according to claim 1 wherein the expression level of NKp46 is determined by flow cytometry.

4. A method for predicting survival time of a patient suffering from acute myeloid leukemia (AML) comprising i) determining in a sample obtained from the patient an expression level of NKp46 and an Hematopoietic Cell Transplant-Co-morbidity Index (HCT-CI) score ii) comparing the expression level and the HCT-CI score determined at step i) with its predetermined reference value and a HCT-CI reference score and iii) providing a good prognosis when the expression level determined at step i) is higher than its predetermined reference value or when the HCT-CI is equal to 0, or providing a bad prognosis when the expression level determined at step i) is lower than its predetermined reference value and the HCT-CI is superior or equal to 1.

5. The method of claim 3, wherein the flow cytometry is performed using monoclonal antibodies conjugated to a detectable label.

6. The method of claim 5, wherein the detectable label is phycoerythrin-cyanine.

7. An analytical method, comprising
obtaining a sample from a patient suffering from acute myeloid leukemia (AML), and
measuring an expression level of NKp46 in said cell or tissue sample.

8. The method according to claim 7, wherein the sample is blood, peripheral blood, serum, plasma or purified NK cells.

9. The method according to claim 7, wherein the patient suffering from AML has been treated by allograft.

10. The method according to claim 7, wherein the expression level of NKp46 is determined by flow cytometry.
